(19) United States
(12) Patent Application Publication
(10) Pub. No.: US 2009/0275795 A1
(43) Pub. Date: Nov. 5, 2009

(54) CONTINENT OSTOMY SYSTEM WITH CHEMICAL NEUROMUSCULAR CONTROL

(75) Inventors: Nicholas Martino, Fernandina Beach, FL (US); John Minasi, Amelia Island, FL (US); James G. Schneider, Chesterfield, MO (US)

Correspondence Address: HUSCH BLACKWELL SANDERS LLP, 190 CARONDELET PLAZA, SUITE 600, ST. LOUIS, MO, 63105-3441, US

(73) Assignee: Zassi Medical Evolutions, Inc., Fernandina Beach, FL (US)

(21) Appl. No.: 12/114,313

(22) Filed: May 2, 2008

(51) Int. Cl. A61F 2/02

(52) U.S. Cl. 600/32

(57) ABSTRACT

An apparatus for providing continence to a gastrointestinal ostomy of a patient, wherein the apparatus includes a lumen sealing device, a chemical neuromuscular control agent, and a mechanism for controlled, localized delivery of the neuromuscular control agent. The lumen sealing device is positioned in contact with the mucosal wall of the intestine and the neuromuscular control agent is delivered to the intestine via the mechanism for controlled, localized delivery, such that the neuromuscular control agent provides an inhibitory effect on peristalsis or smooth muscle contraction and relaxation cycles in the intestine to thereby arrest the advancement of the contents of the intestine.

### CROSS-REFERENCE TO RELATED APPLICATIONS

None

### STATEMENT REGARDING FEDERALLY SPONSORED RESEARCH

Not Applicable.

### APPENDIX

Not Applicable.

### FIELD OF THE INVENTION

The present invention relates generally to continence devices, and more particularly to continent ostomy devices. What is described is a method and apparatus to provide continence to a gastrointestinal ostomy utilizing a lumen sealing device in conjunction with a chemical neuromuscular control agent to inhibit peristalsis or smooth muscle contraction and relaxation cycles of the intestinal muscles.

### BACKGROUND OF THE INVENTION

An ostomy is a surgically-made opening in the body. Ostomies may be of a variety of types including, but not limited to, ileostomies, colostomies and urostomies. Although the discussion below will usually describe the invention with reference to the ostomy resulting from a colostomy procedure, it is to be understood that the invention can be applied to other types of ostomies as well. Further, although the discussion generally considers human patients, the structures described and claimed herein can be useful in non-human mammals as well.

A variety of medical conditions can lead to ostomy surgery, but the four most prevalent are colorectal cancer, diverticulitis, Crohn's Disease and ulcerative colitis. Ostomy surgery for these conditions generally requires the resection of part or the entire colon and/or rectum and the subsequent diversion of the colon or small bowel via an ostomy wherein the end of the remaining healthy portion of the colon or small bowel is brought through the abdominal wall, inverted on itself and sutured in place to form a stoma S. During inactive periods, the tissue pulls together, rather like puckered lips. When waste is expelled the stoma stretches to permit the waste to pass. Although it expands and contracts, the stoma does not have the firm muscle control of the anal sphincter. The muscles M of the remaining segment of colon remain intact and continue to function in a coordinated manner to effectively advance stools distally toward the stoma. However, while the colonic muscles remain relatively unaffected, the storage capacity naturally provided by the rectum and the muscle control provided by the anal sphincters are no longer available. Because of the loss of these key functions, the individual is rendered fecally incontinent (i.e., unable to control the time and place of waste evacuation) and must defecate into an ostomy bag glued to their abdomen around their stoma for as long as they have the colostomy.

Fecal management in this manner is generally effective and is the current standard of care for individuals with colostomies. However, the use of ostomy bags often results in these individuals experiencing a variety of problems not ordinarily experienced by the general public (i.e., those with normal defecatory anatomy). These problems include leakage of intestinal gas, mucus, and waste, such as liquid and solid fecal material through the adhesive seal holding the ostomy bag in place. Such leakage not only causes unpleasant odors, but also leads to health problems, such as necrosis of the tissue surrounding the stoma site. The rate of leakage occurrence increases as the bag fills and the resulting weight pulls on the adhesive seal between the bag and the abdominal wall. Even when ostomy bags perform optimally, the fear of leakage, odor and the stigma associated with wearing the ostomy bag can have negative effects on the individual's quality of life, particularly their social and psychological well being.

The known art has made a variety of attempts to address these problems with various non-bag devices, without complete success. A number of barrier devices (e.g. foam plugs, catheter ports and inflatable sealing membranes) have been developed which essentially plug or seal the stoma until the user is ready to evacuate. To one degree or another, each of these devices was unable to maintain a safe and/or reliable seal with the intestinal lumen in which they resided, and resulted in leakage of waste around the device, device expulsion and/or tissue damage. Much of the lack of success of these devices can be attributed to the smooth muscle characteristics of the intestinal lumen. Generally, the nature of a smooth muscle lumen is to accommodate to any chronic bolus present within the lumen. In the case where the bolus is a stationary sealing structure, increases in the circumference of the intestinal lumen, as it accommodates, can contribute to leakage of luminal contents around the sealing structure.

To date, only one non-bag ostomy sealing device for the management of colostomies has been made commercially available. The Conseal® Plug made by Coloplast® is a tissue lumen sealing device having an adhesive base plate and a foam plug that prevents the passage of feces (fluid or solid). The plug is supplied in a compressed state within a water-soluble film. The film disintegrates within a few seconds of insertion and the plug expands to its natural size to seal the stoma. The plug is removed to allow for fecal evacuation, after which a new plug is inserted. Although commercialized, the Conseal Plug suffered from some of the short comings noted above specifically incidents of leakage and device expulsion.

The technical challenges faced in attempting to seal the gastrointestinal ostomy are better understood by reviewing the normal chemical, electrical and mechanical physiological mechanisms effecting colonic motility (i.e., the involuntary muscular activity of the colon which coordinates the movement of digesting materials towards the anus).

### The Enteric Nervous System

The nervous system of the human body (and, for that matter, all mammals) has a profound influence on all digestive processes including colonic motility. Some of this control originates from connections between the central nervous system and the gastrointestinal tract, but just as importantly, the gastrointestinal tract is endowed with its own local nervous system referred to as the enteric nervous system.

The principal components of the enteric nervous system are two networks or plexuses of neurons (the myenteric plexus and the submucosal plexus), both of which are embedded in the wall of the gastrointestinal tract. These enteric neurons secrete an array of chemical neurotransmitters that permit nerve signals to bridge the gap between nerve cells. Certain neurotransmitters are excitatory in nature, stimulating smooth muscle contractions, while others are inhibitory in nature, stimulating smooth muscle relaxation.

While the enteric nervous system can and does function autonomously, normal gastrointestinal function requires communication links between the enteric nervous system and the central nervous system. These links take the form of parasympathetic and sympathetic nerve fibers that connect either the central and enteric nervous systems or connect the central nervous system directly with the gastrointestinal tract. Through these cross connections, the gastrointestinal tract can provide sensory information to the central nervous system, and the central nervous system can affect gastrointestinal function. One example of the nervous interconnections within the gastrointestinal tract is the gastrocolic reflex, where distension of the stomach stimulates evacuation of the colon.

In general, parasympathetic nerve stimulation is excitatory in nature, causing contraction of gastrointestinal smooth muscle and increased gastrointestinal secretion and motor activity. Conversely, sympathetic nerve stimuli typically inhibit these activities.

### Colonic Motility and Smooth Muscle

The colon is a dynamic luminal organ. Muscles located on the exterior run along the length of the colon, extending and retracting the colon like a rubber band. These muscles contribute to a muscle action called haustral churning which facilitates mixing, fluid absorption and particle cohesion. Interior muscles wrap around the colon in circular bands that distend and contract the colon wall in an action that is similar to opening and closing a fist. Working in concert, these muscles contribute to the principal type of motility called peristalsis (i.e., a distinctive pattern of smooth muscle contraction and relaxation that propels digesting materials distally toward the anus). Ultimately, the peristalsis advances stool into the rectum. When stool fills the rectum, the elastic quality of the wall permits the rectum to expand, creating a sac to accommodate stools just prior to defecation.

All muscles in the colon wall are smooth muscle which has properties distinctly different from skeletal muscle. Unlike skeletal muscle, smooth muscle is not under voluntary control. Smooth muscle fibers are arranged in intertwined, indistinct bundles, aligned in circular and longitudinal layers. Individual smooth muscle fibers are connected to neighboring smooth muscle cells by gap junctions, which allow these cells to be electrically coupled. The important consequence of this electrical coupling is that when an area of smooth muscle becomes depolarized, that depolarization spreads outward through adjacent sections of smooth muscle resulting in a well-coordinated contraction of, for example, an entire ring of circular smooth muscle.

### Electrophysiology of Gastrointestinal Smooth Muscle

Normal gastrointestinal motility results from coordinated contractions of smooth muscle, which in turn derive from two basic patterns of electrical activity across the membranes of smooth muscle cells—slow waves and spike potentials.

Like other excitable cells, gastrointestinal smooth muscle cells maintain an electrical potential difference across their membranes. However, in contrast to nerves and other types of muscle cells, the membrane potential of smooth muscle cells fluctuates spontaneously. Because the cells are electrically coupled, these fluctuations in membrane potential spread to adjacent sections of muscle, resulting in what are called “slow waves”—waves of partial depolarization in smooth muscle that sweep along the gastrointestinal tract for long distances. The frequency of slow waves depends on the section of the gastrointestinal tract; in the small intestine they occur 10 to 20 times per minute and in the large intestine 3 to 8 times per minute. Slow wave activity appears to be a property intrinsic to smooth muscle and dependent on nervous stimuli.

Importantly, slow waves are not action potentials and by themselves do not induce contractions. Rather, they coordinate muscle contractions in the gastrointestinal tract by controlling the appearance of a second type of depolarization event referred to as “spike potentials,” which occur only at the crests of slow waves. Spike potentials are true action potentials that induce muscle contraction. They result when a slow wave passes over an area of smooth muscle that has been primed by exposure to neurotransmitters released in their vicinity by neurons of the enteric nervous system. The neurotransmitters are released in response to a variety of local stimuli, including distension of the wall of the gastrointestinal tract and serve to “sensitize” the muscle by making its resting membrane potential more positive.

It is thus apparent how a particular pattern of motility is achieved. For example, when a large bolus (e.g., ingested food) enters the intestine the bolus distends the intestine, stretching its walls. This stretching stimulates nerves in the wall of the intestine to release neurotransmitters into smooth muscle at the site of distention and the membrane potential of that section of muscle becomes “more depolarized.” When a slow wave passes over the section of smooth muscle exposed to the neurotransmitters, spike potentials form and muscle contraction results; the contraction moves around and along the intestine in a coordinated manner because the muscle cells are electrically coupled through gap junctions. These coordinated muscle contractions work to mix and propel digesting materials distally.

In view of the various shortcomings of the known art, the primary goal of the present invention is to overcome the loss of the normal physiological mechanisms associated with the anatomical derangements of a colostomy procedure and the shortcomings of previous non-bag ostomy management devices, and in so doing allow the individual to be effectively continent.

### SUMMARY OF THE INVENTION

The present invention utilizes a lumen sealing device for creating a sealing surface with the mucosal wall of the intestine I, in conjunction with a chemical neuromuscular control agent. The neuromuscular control agent is delivered in an amount effective to locally inhibit peristalsis or smooth muscle contraction and relaxation cycles in the intestine for the purpose of arresting the advancement of intestinal contents in a segment of the intestine. Depending on the selection of neuromuscular control agent as well as the delivery mechanism utilized, the inhibitory affect can be short term, for example, remaining effective for a period of 12 hours or less; long term, remaining in effect for periods greater than 12 hours and up to multiple days; or chronic, remaining in effect for weeks or months. In addition to the neuromuscular control agent, the lumen sealing device serves to prevent passive drainage (i.e., due to gravity or intra-abdominal pressure) of less-than-solid waste products (e.g., liquid or semi-liquid stools) or leakage of exudate from the stoma by blocking and/or capturing the intestinal contents.

Generally, the effect of the neuromuscular control agent is temporary and is required for a duration of time equal to or greater than the duration of time the luminal sealing device remains in place. When the effect of the neuromuscular control agent diminishes or ends, the sealing structure may be removed, and the normal peristalsis or smooth muscle contraction and relaxation cycles will return to advance stools distally for evacuation through the stoma S (although evacuation may also be facilitated by any one of various ways, e.g., irrigation, emulsification, physical capture and removal, the use of motility stimulant agents and electrical muscle stimulation. Therefore, when used in conjunction, the luminal sealing device and the neuromuscular control agent of the present invention provide a method of continence to the gastrointestinal ostomy O.

The present invention offers a number of key advantages over the known art. The apparatus described and claimed herein allows the sealing device to maintain an effective seal with the mucosal wall of the intestine, i.e., the expansion and contraction of the intestinal lumen due to the contraction and relaxation cycles of the intestinal muscles are reduced or eliminated, allowing the sealing device to maintain contact with the mucosal wall of the intestine. The new apparatus also reduces prograde and retrograde migration of the sealing device away from the desired sealing location, i.e., the propelling effect of the contraction and relaxation cycles of the intestinal muscles on the sealing device is reduced or eliminated. Moreover, the new device reduces or eliminates inadvertent expulsion of the sealing device from the ostomy.

The present apparatus also reduces tissue erosion at the sealing surface between the sealing device and the intestinal lumen, i.e., the eroding effect of continuous muscle contraction and relaxation cycles on the sealing device is reduced or eliminated.

Also, in the case where a neuromuscular control agent is utilized to maintain the intestinal muscles in a chronic state of paralysis, the new apparatus allows the creation of a proximal reservoir, i.e., over time the muscles proximal to the sealing device will atrophy and will gradually begin to accommodate a larger volume of stool and will mature into a neo-rectum providing “reservoir continence.”

Accordingly, the invention is, briefly, an apparatus for providing continence to a gastrointestinal ostomy of a patient. The apparatus includes a lumen sealing device, a chemical neuromuscular control agent, and a mechanism for controlled, localized delivery of the neuromuscular control agent. The lumen sealing device is positioned in contact with the mucosal wall of the intestine and the neuromuscular control agent is delivered to the intestine via the mechanism for controlled, localized delivery, such that the neuromuscular control agent provides an inhibitory effect on peristalsis or smooth muscle contraction and relaxation cycles in the intestine to thereby arrest the advancement of the contents of the intestine.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a longitudinal sectional view of an apparatus for providing continence to a gastrointestinal ostomy, shown in place in an ostomy, the apparatus being constructed in accordance with and embodying the present invention and showing a plug type sealing device with a neuromuscular control agent incorporated into the coating on the sealing device.

FIG. 2 is a longitudinal sectional view of an apparatus for providing continence to a gastrointestinal ostomy, shown in place in an ostomy, the apparatus being constructed in accordance with and embodying the present invention and showing a catheter and occluding plug type sealing device with neuromuscular control agent incorporated into the coating on the sealing device.

FIG. 3 is a longitudinal sectional view of an apparatus for providing continence to a gastrointestinal ostomy, shown in place in an ostomy, the apparatus being constructed in accordance with and embodying the present invention and showing a stoma sealing membrane type sealing device with neuromuscular control agent incorporated into the coating on the sealing device.

FIG. 4 is a longitudinal sectional view of an apparatus for providing continence to a gastrointestinal ostomy, shown in place in an ostomy, the apparatus being constructed in accordance with and embodying the present invention and showing an absorbent tampon type sealing device with neuromuscular control agent incorporated into the sealing device material per se.

FIG. 5 is a longitudinal sectional view of an apparatus for providing continence to a gastrointestinal ostomy, shown in place in an ostomy, the apparatus being constructed in accordance with and embodying the present invention and showing an absorbent pad type sealing device with neuromuscular control agent incorporated into the sealing device material per se.

FIG. 6 is a longitudinal sectional view of an apparatus for providing continence to a gastrointestinal ostomy, shown in place in an ostomy, the apparatus being constructed in accordance with and embodying the present invention and showing an absorbent tampon type sealing device with neuromuscular control agent incorporated into an elongated, polymer coating which dissolves in the ostomy.

FIG. 7 is a longitudinal sectional view of an apparatus for providing continence to a gastrointestinal ostomy, shown in place in an ostomy, the apparatus being constructed in accordance with and embodying the present invention and showing a plug type sealing device comprising an administration lumen for administration of the neuromuscular control agent through the sealing device.

FIG. 8 is a longitudinal sectional view of a further embodiment of the invention of FIG. 8, showing a neuromuscular control agent incorporated into the coating on the sealing device.

FIG. 9 is a longitudinal sectional view of a further embodiment of the invention of FIG. 8, showing an agent reservoir incorporated into an exterior abdominal faceplate.

FIG. 10 is a longitudinal sectional view of an apparatus for providing continence to a gastrointestinal ostomy, shown in place in an ostomy, the apparatus being constructed in accordance with and embodying the present invention and showing a plug type sealing device comprising a microporous balloon as a reservoir for the neuromuscular control agent.

### DETAILED DESCRIPTION OF THE PREFERRED EMBODIMENTS

The following description of the preferred embodiment(s) is merely exemplary in nature and is in no way intended to limit the invention, its application, or uses.

In its basic form, the new apparatus for providing continence to a gastrointestinal ostomy O, generally designated 10, includes a luminal sealing device 12 contactable with the mucosal wall W of the intestine I in a gastrointestinal ostomy O, a chemical neuromuscular control agent 52 having an inhibitory effect on peristalsis or smooth muscle contraction and relaxation cycles, and an administration mechanism 14 for controlled, localized delivery of the neuromuscular control agent 52 to the intestine I.

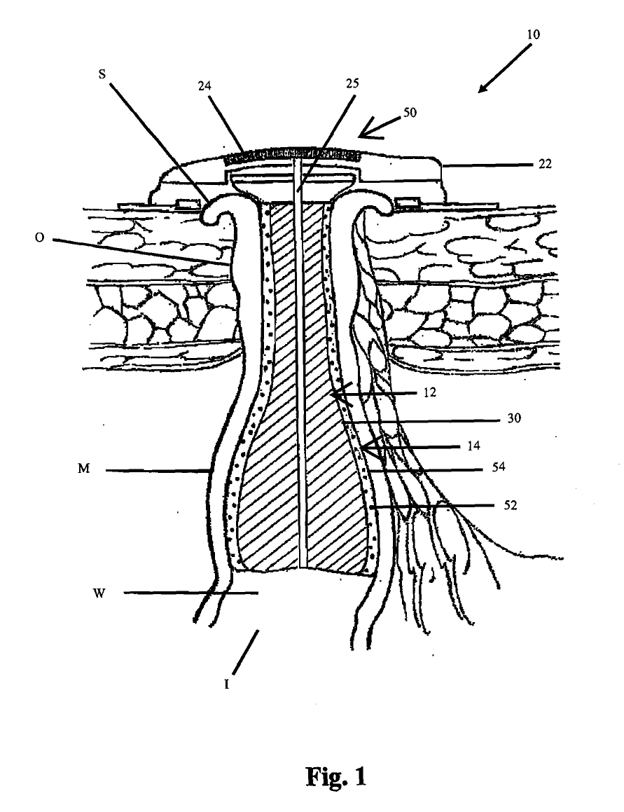

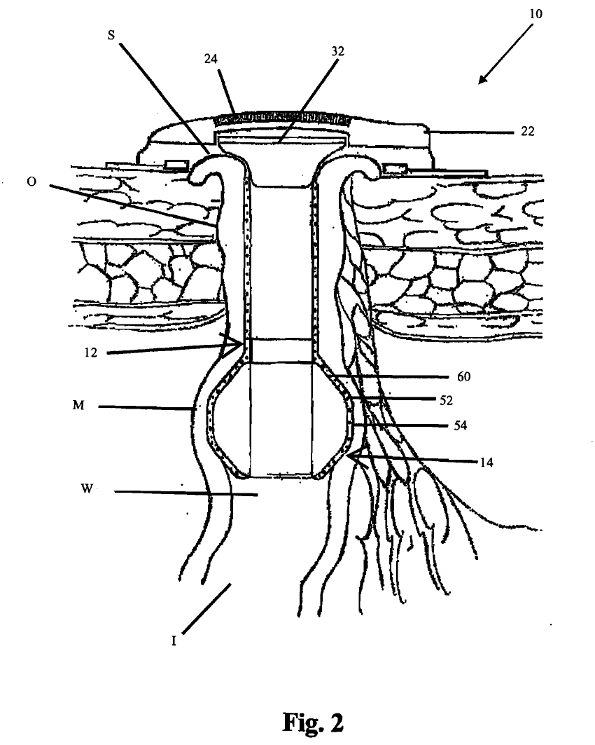

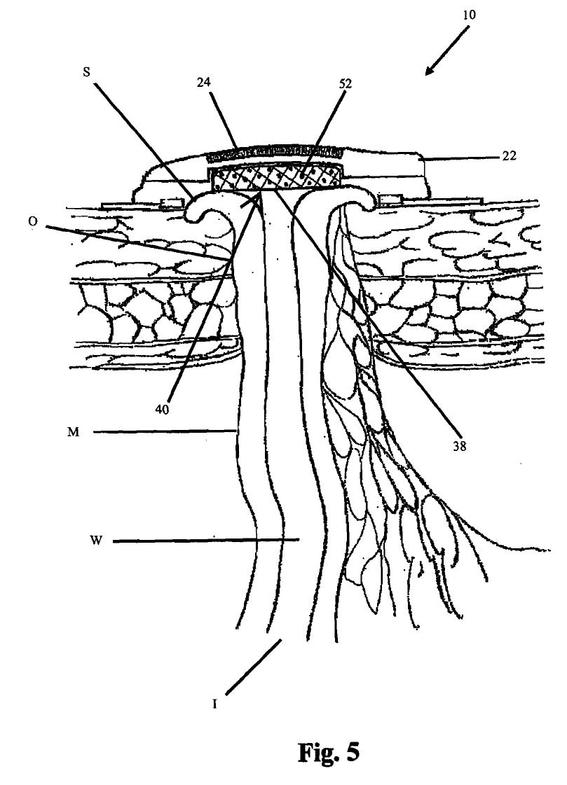

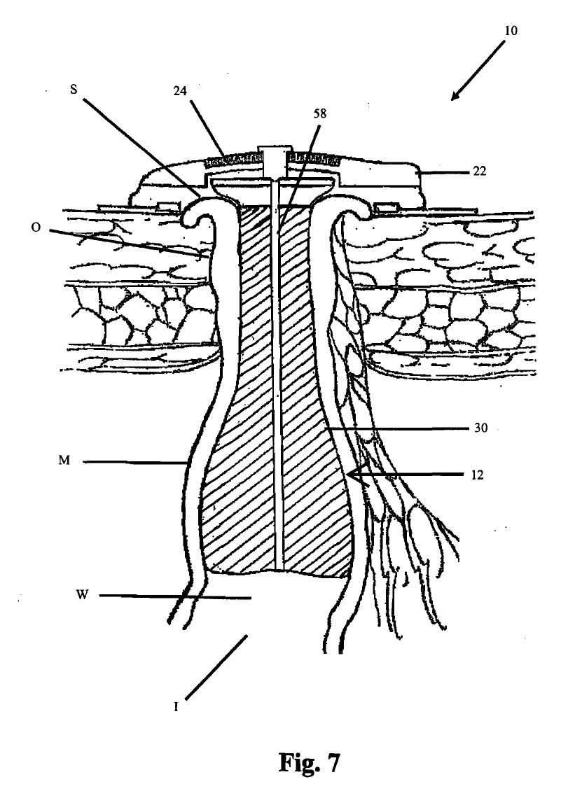

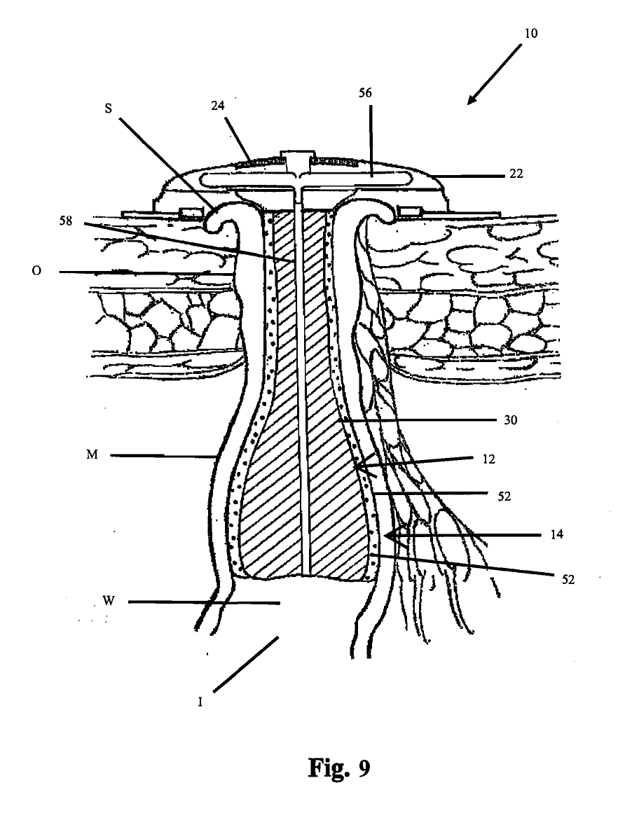

All or part of the apparatus 10 can be located inside the patient's body. Alternatively, all or part of the apparatus 10 can be located outside the patient's body. For example, the lumen sealing device 12 may be located inside the patient's body, such as, a plug residing within the intestinal lumen (e.g., FIG. 1), or, alternatively, the lumen sealing device 12 may be located outside the patient's body, such as, an absorbent pad placed against the stoma S (e.g., FIG. 5). The neuromuscular control agent 52 may be located inside the patient's body, such as, incorporated into the sealing device 12 per se (e.g., FIG. 1), or alternatively, the neuromuscular control agent 52 may be located (stored) outside the patient's body and delivered therefrom, such as, integrated into an exterior abdominal faceplate (e.g., FIG. 9). The administration mechanism 14 for controlled, localized delivery of the neuromuscular control agent 52 may be located inside the patient's body, such as, a coating on the sealing device 12 incorporating the neuromuscular control agent 52 (e.g., FIG. 2), or alternatively, the mechanism may be located outside the patient's body, such as, an agent administration lumen 58 integrated into an exterior abdominal faceplate (e.g., FIGS. 7-9).

The lumen sealing device 12 of the present invention prevents unintentional passage of waste products and exudates present in the intestinal lumen to outside the patient's body. For the purpose of the present invention, a lumen sealing device 12 is defined as any structure, or structure coating, in contact with the mucosal wall W of the intestine I (internal and/or external to the patient) for the purpose of providing a seal sufficient to prevent the elimination of at least solid and liquid substances from the ostomy O.

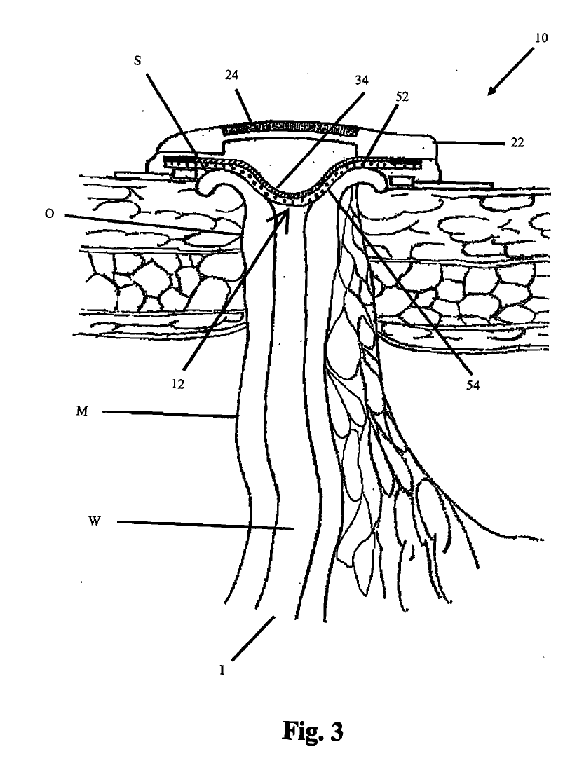

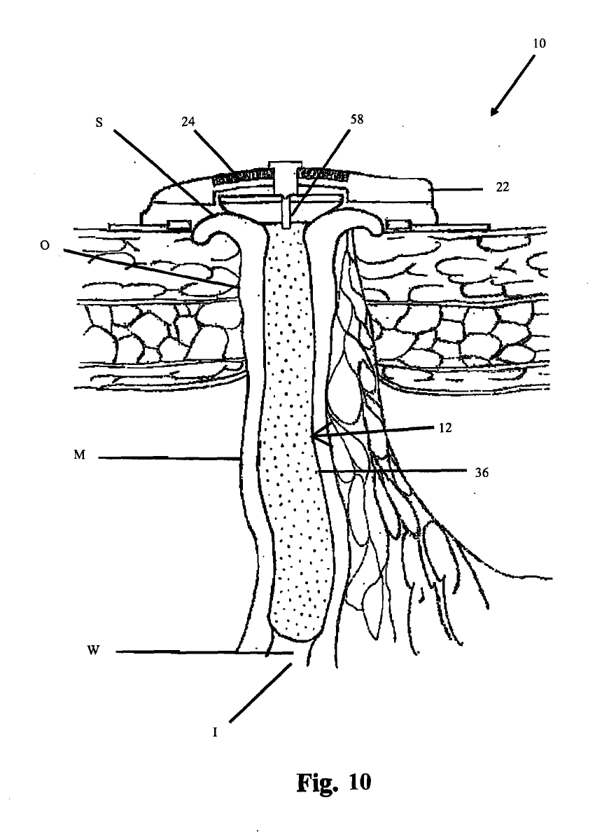

In one group of embodiments, those examples shown in FIGS. 1, 2, 3, 7, 8, 9, and 10, the sealing device 12 is made of a generally non-absorbent, water-insoluble material, for example, polyurethane, and the seal is accomplished via surface-to-surface contact between the mucosal wall W of the intestine I and the sealing device 12. Examples include: polyurethane closed-cell foam plugs 30 (e.g., FIG. 1 and 7-9), polyurethane catheters 60 with removable occluding plugs 32 (e.g., FIG. 2), polyurethane stoma sealing membranes 34 (e.g., FIG. 3) and polyurethane microporous membrane balloons 36 (e.g., FIG. 10).

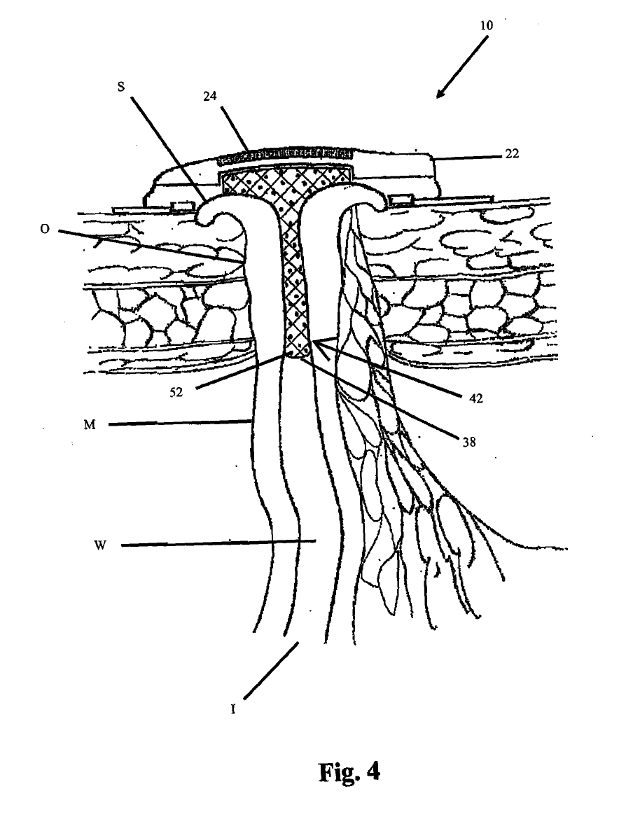

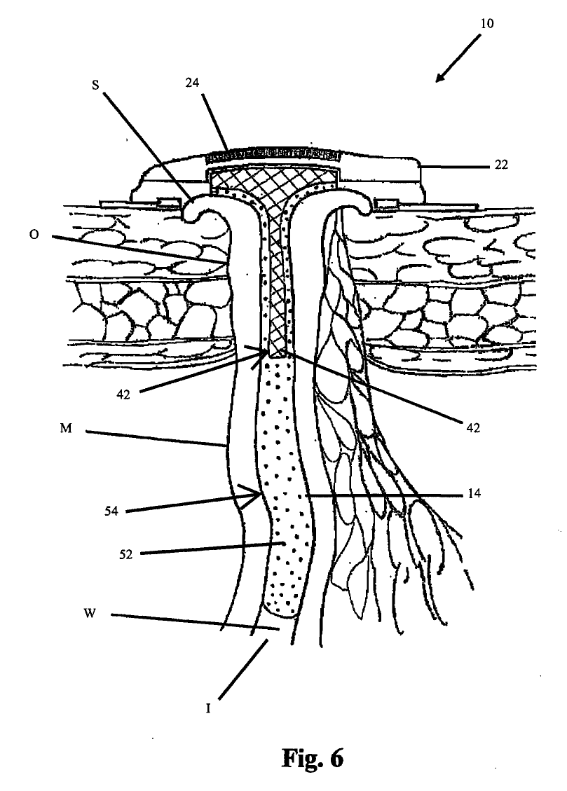

In another group of embodiments (FIGS. 4-6), the lumen sealing device 12 is made of a generally absorbent material 38, for example, polyurethane foam, and the seal is accomplished by absorbing the waste products and exudates present in the colonic lumen that contact the sealing device 12. Examples include: polyurethane open-cell foam tampons 42 (FIGS. 4 and 6) and polyurethane open-cell foam pads 40 (FIG. 5).

Generally, the lumen sealing devices 12 remain in place for the period of time continence is desired and are selectively removed for evacuation of the bowel of the patient, after which the sealing device 12 is put back in place or disposed of and a new sealing device 12 is put in place, or alternatively, the patient returns to the use of their ostomy bag for some duration. However, in the case of the catheter 60 with a removable plug 32, shown in FIG. 2, the catheter portion can remain indwelling and the plug separately removed for bowel evacuation. The plug can then be re-inserted or disposed of and replaced, if preferred or necessary.

If preferred, the lumen sealing device 12 may incorporate a mechanism to vent flatus, such as a venting channel 25, for example, as shown in FIG. 1, which allows flatus to pass through the lumen sealing device 12. Alternatively, the lumen sealing device 12 may have irregular surfaces such as protrusions, for example, which allow flatus to pass around the lumen sealing device 12 while still maintaining a seal with the mucosal wall W of intestine I. If desired, apparatus 10 may also incorporate a deodorizing element 24 within cover 22 and above (exterior of) the distal end 50 of lumen sealing device 12 when cover 22 is in the closed position shown.

The neuromuscular control agent 52 of the present invention inhibits peristalsis or muscle contraction and relaxation cycles in the smooth muscles M of the intestine I for the purpose of arresting the advancement of intestinal contents toward the stoma S.

A preferred neuromuscular control agent 52 is L-menthol. L-menthol inhibits gastrointestinal peristalsis or smooth muscle contraction and relaxation cycles through a calcium channel blocking effect. L-menthol is a main component of peppermint oil or mentha oil and is obtained by steam distillation of a plant, for example, Mentha piperita or Mentha arvensis, which typically contain 30% or more by weight of L-menthol. The L-menthol-containing material employed may be peppermint oil or mentha oil as is, but highly purified L-menthol obtained for example by fractional distillation of peppermint oil or mentha oil can also be employed preferably. More preferably, L-menthol of a purity of 90% or more by weight is employed. Recently, an L-menthol product is produced by synthesis and is also employed.

Additional examples of known neuromuscular control agents 52 applicable to the present invention include: botulinum toxin, tetanus toxin, tetrodotoxin, saxitoxin, batrachotoxin, hemicholinium, magnesium ions, 4-aminopyridine, curare alkaloids, snake alpha-toxins, calcium inhibitors, decamethonium, veratrine, quinine, metabolic poisons, dantrolene, methacholine, atropine, trimethaphan, doxyrubicin, anticholinergic agents and compounds derived from plants. All these compounds are considered as potentially useful in the new apparatus and method. Further, other useful compounds may be developed or discovered in nature and would thus also be considered within the meaning of the invention. Still further, suitable neuromuscular control agents 52 may be used alone or in combination with other neuromuscular control agents 52.

The effects on the patient of the neuromuscular control agent 52 can diminish or end spontaneously within a known period of time or the effects can be reversed by the use of appropriate counter-active agents. For example, in the case where botulinum toxin is used as the neuromuscular control agent 52, the effects of botulinum toxin can be reversed by the administration of antitoxin to the bacterium, clostridium botulinum, or by the administration of anticholinesterase inhibitors such as physostigmine.

The administration mechanism 14 of the present invention provides a controlled, localized delivery of the neuromuscular control agent 52 to a segment of the patient's intestine I, in an amount effective to inhibit peristalsis or muscle contraction and relaxation cycles in the smooth muscles M of the intestine I.

The preselected neuromuscular control agent 52 may be administered to the intestine I in a variety of ways. In FIGS. 1-10, the administration mechanism 14 may be that the sealing device 12 is used to administer a dose of agent locally to the mucosal surface of the intestine I. In various examples (e.g., FIGS. 1, 2, 3, 6, 8, and 9), a composition of rapidly dissolving, water soluble, film-forming polymer 54 combined with a neuromuscular control agent 52 is coated over at least a portion of the sealing device 12 and is used to deliver the neuromuscular control agent 52 to the mucosal membrane of the intestine I. Upon exposure to the intestinal mucosa, the film forming agent will hydrate substantially immediately to form a thin coating on the moist surface of the mucosal membrane and then disintegrate and, or, dissolve (preferably within 1 to 600 seconds, more preferably within 1 to 60 seconds) entrapping the neuromuscular control agent 52 against the mucosal surface to provide prolonged neuromuscular control efficacy. A preferred water soluble, film-forming polymer agent is pullulan, a biodegradable, polysaccharide polymer, in an amount up to 99 wt %, preferably from 5 to 45 wt %, more preferably from 15 to 25 wt %. Additional examples of water soluble, film-forming polymer agents applicable to the present invention include: hydroxypropylmethyl cellulose, methyl cellulose, ethyl cellulose, hydroxyethyl cellulose, hydroxypropyl cellulose, polyvinyl pyrrolidone, carboxymethyl cellulose, polyvinyl alcohol, sodium alginate, polyethylene glycol, polyethylene oxides, pluronics, tetronics, xantham gum, tragacanth gum, guar gum, acacia gum, gum ghatti, okra gum, karaya gum, locust bean gum, tara gum, quince seed gum, hyaluronic acid, Arabic gum, polyacrylic acid, methylmethacrylate copolymer, carboxyvinyl polymer, amylase, high amylase starch, hydroxypropylated high amylase starch, dextran, dextrin, pectin, chitin, chitosan, levan, elsinan, collagen, gelatin, zein, gluten, soy protein isolate, whey protein isolate, casein and mixtures thereof. In addition to the film-forming polymer agent, other ingredients used to make the composition may include: plasticizing agents, surfactants, stabilizing agents, emulsifiers, thickening agents, binding agents, preservatives, triglycerides, polyethylene oxide and propylene glycol.

In a variation of the embodiment (e.g., FIG. 6), the dosage form of the composition may be cast onto the sealing device 12 in an elongated shape for the purpose of increasing the length of intestine I affected by the neuromuscular control agent 52.

The amount of specific neuromuscular control agent 52 provided in each dose will obviously be dependent on the dose needed to provide an inhibitory effect on peristalsis or smooth muscle contraction and relaxation cycles in the targeted segment of intestine I. Furthermore, the amount provided may be adjusted to deliver a predetermined dose over a predetermined period of time. The concentration of neuromuscular control agent 52 in accordance with the invention may be up to 99 wt %, but is typically in the range of 0.1 to 50 wt %. Typical doses which can be delivered per dosage form are in the range of 10 micrograms to 900 milligrams, however, in the case where the neuromuscular control agent 52 is a plant compound, (e.g., herbs), multiple grams may be delivered per dosage form.

In another example of the sealing device 12 being used to administer a dose of neuromuscular control agent 52 locally to the mucosal surface of the intestine I, a neuromuscular control agent 52 is distributed into the sealing device material itself, where the sealing device material is, for example, a polymer or polymeric matrix (e.g., FIGS. 4 and 5), wherein fractional amounts of the neuromuscular control agent 52 can be released from the polymer or polymeric matrix to provide prolonged neuromuscular control efficacy.

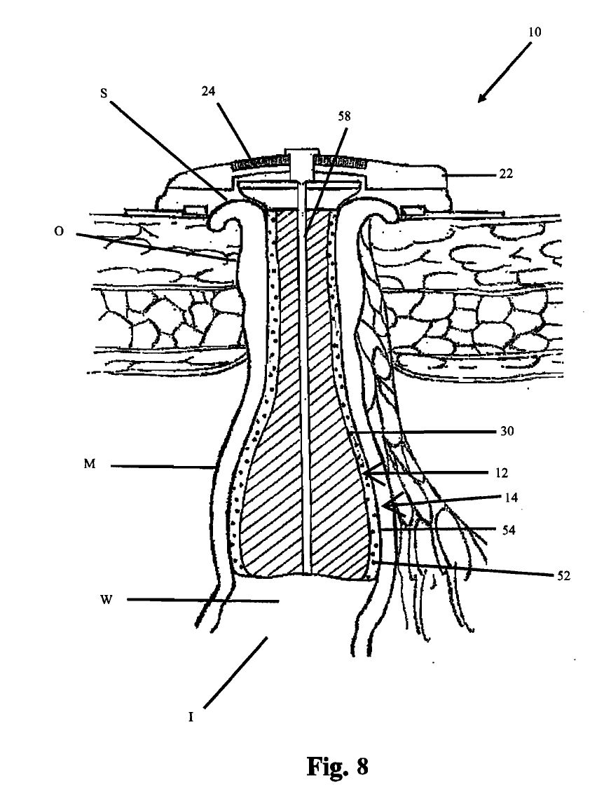

Other examples of the sealing device 12 being used to administer a dose of neuromuscular control agent 52 locally to the mucosal surface of the intestine I include: a sealing device 12 comprising a microporous balloon 36 as an agent reservoir 56 to retain and dispense the neuromuscular control agent 52 (e.g., FIG. 10), and an agent administration lumen integrated into the sealing device 12 (e.g., FIGS. 7-10). FIG. 8 is a further embodiment of FIG. 7 with the addition of a neuromuscular control agent 52 incorporated into the coating on the sealing device 12. FIG. 9 is a further embodiment of FIG. 8 with the addition of an agent reservoir 56 connectable to the administration lumen 58 and incorporated into an exterior abdominal faceplate. All four embodiments allow for the periodic re-administration of agent to the intestine I.

Depending upon the type selected, administration mechanism 14 can deliver the neuromuscular control agent 52 to the intestinal serosa, mucosa, submucosa or muscularis layers, as desired. The dose and frequency of administration can vary depending on muscle mass and length of time the effect of the neuromuscular control agent 52 is required.

Generally, the effect of the neuromuscular control agent 52 is temporary and is required for a duration of time equal to or greater than the duration of time the sealing device 12 remains in place. However, in the case where a return of peristalsis or muscle contraction and relaxation cycles is desired to assist in expelling the sealing device 12 from the body, the neuromuscular control agent 52 can be administered in a manner so that its effect diminishes or ends before the sealing device 12 is removed from the body. Also, if the sealing device 12 is required for an extended duration of time, the effect of the neuromuscular control agent 52 can be extended by periodically re-administering the agent to the intestine I via the sealing device 12 itself, such as, via an administration lumen 58.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. An apparatus for providing continence to a gastrointestinal ostomy of a patient, comprising:
a lumen sealing device contactable with the mucosal wall of the intestine in a gastrointestinal ostomy;
a chemical neuromuscular control agent capable of providing an inhibitory effect on peristalsis or smooth muscle contraction and relaxation cycles; and
a mechanism for controlled, localized delivery of the neuromuscular control agent to the intestine;
wherein the neuromuscular control agent inhibits peristalsis or smooth muscle contraction and relaxation cycles in the patient's intestine to thereby arrest the advancement of intestinal contents in a segment of the intestine.

2. The apparatus of claim 1, wherein the lumen sealing device is a plug in contact with the mucosal wall of the patient's intestine.

3. The apparatus of claim 1, wherein the lumen sealing device is a selectively occludeable catheter in contact with the mucosal wall of the patient's intestine.

4. The apparatus of claim 1, wherein the lumen sealing device is a stoma sealing membrane in contact with the mucosal wall of the patient's intestine.

5. The apparatus of claim 1, wherein the lumen sealing device is an absorbent tampon in contact with the mucosal wall of the patient's intestine.

6. The apparatus of claim 1, wherein the lumen sealing device is an absorbent pad in contact with the mucosal wall of the patient's intestine.

7. The apparatus of claim 1, wherein the lumen sealing device further comprises a vent to permit release of flatus via the ostomy.

8. The apparatus of claim 1, wherein the neuromuscular control agent is a gastrointestinal motility suppressant agent selected from the group consisting of muscle relaxants, anticholinergic agents, calcium channel blockers, neuromuscular blocking agents, opioids, neurotoxins, neurotransmitters, hormonal agents, antidiarrheal agents, compounds derived from plants, and combinations thereof.

9. The apparatus of claim 1, wherein the mechanism for controlled, localized delivery is a soluble polymer.

10. The apparatus of claim 1, wherein the mechanism for controlled, localized delivery is an insoluble polymer matrix.

11. The apparatus of claim 1, wherein the mechanism for controlled, localized delivery is a porous polymer membrane.

12. The apparatus of claim 1, wherein the mechanism for controlled, localized delivery is an administration lumen.

13. A method for providing continence to a gastrointestinal ostomy of a patient; comprising:
providing a patient having a gastrointestinal ostomy with an apparatus for providing continence, the apparatus having a lumen sealing device contactable with the mucosal wall of the intestine in the gastrointestinal ostomy; a chemical neuromuscular control agent capable of providing an inhibitory effect on intestinal peristalsis or smooth muscle contraction and relaxation cycles; and a mechanism for controlled, localized delivery of the neuromuscular control agent to the intestine;
the neuromuscular control agent inhibits peristalsis or smooth muscle contraction and relaxation cycles in the patient's intestine, thereby arresting the advancement of intestinal contents in a segment of the intestine.

14. The method of claim 13, and further comprising administering the neuromuscular control agent via the sealing device.

15. The method of claim 13, and further comprising administering the neuromuscular control agent to the intestinal mucosa of the patient.

16. The method of claim 13, and further comprising administering the neuromuscular control agent to the intestinal serosa.

17. The method of claim 13, and further comprising administering the neuromuscular control agent to the intestinal submucosa.

18. The method of claim 13, and further comprising administering the neuromuscular control agent to the intestinal muscularis.

19. The method of claim 13, and further comprising administering the neuromuscular control agent to the area of contact between the sealing device and the mucosal wall of the patient's intestine.

20. The method of claim 13, and further comprising administering the neuromuscular control agent distal to the sealing device.

21. The method of claim 13, and further comprising administering the neuromuscular control agent proximal to the sealing device.
